(19) United States
(12) Patent Application Publication
(10) Pub. No.: US 2020/0022449 A1
(43) Pub. Date: Jan. 23, 2020

(54) SENSOR INSERT FOR A SHOE

(71) Applicant: NURVV LIMITED, Twickenham (GB)

(72) Inventors: Jason Roberts, Twickenham (GB); Grant Trewartha, Derry Hill (GB); Haim Geva, London (GB); Giles Tongue, West Byfleet (GB); Kemal Dervish, Welwyn Garden City (GB)

(21) Appl. No.: 16/513,299

(22) Filed: Jul. 16, 2019

(30) Foreign Application Priority Data
Jul. 18, 2018 (GB) 1811722.6

(51) Int. Cl. A43B 3/00; A43B 17/00; B32B 25/08; B32B 25/14; B32B 7/12; A61B 5/103; A61B 5/00

(52) U.S. Cl. CPC A43B 3/0005; A43B 17/006; B32B 25/08; B32B 25/14; B32B 7/12; A61B 2562/227; A61B 5/6807; B32B 2437/02; A61B 2562/0247; A61B 2562/046; A61B 5/1038

(57) ABSTRACT

A senor insert for a shoe includes a flexible planar portion having a shape of an inner sole for a shoe. The flexible planar portion includes a central layer containing a plurality of pressure sensors for sensing the pressure applied by the foot and electrical connections to the sensors; a lower layer having a lower face arranged to face the sole of a shoe and having a first coefficient of friction; and an upper layer having an upper face via which a foot is arranged to impact the insert and having a second coefficient of friction which is lower than the first coefficient of friction.

### CROSS-REFERENCE TO RELATED APPLICATIONS

This application claims the benefit of United Kingdom Patent Application No. GB1811722.6, filed Jul. 18, 2018, the entire disclosure of which is incorporated herein by this reference.

### STATEMENT REGARDING FEDERALLY SPONSORED RESEARCH OR DEVELOPMENT

Not applicable.

### THE NAMES TO PARTIES TO A JOINT RESEARCH AGREEMENT

Not applicable.

### INCORPORATION-BY-REFERENCE OF MATERIAL SUBMITTED ON A COMPACT DISC

Not applicable.

### BACKGROUND OF THE INVENTION

### Field of the Invention

The present invention relates to a sensor insert for a shoe.

### Description of the Related Art

Such a sensor insert is known, for example, from earlier application GB2549513.

This comprises a generally planar flexible portion having the size and shape of the inner sole of a shoe. The sensor insert comprises a plurality of pressure resistors which are arranged across the sensor insert in order to detect the pressure of the foot of a user wearing the shoe. Such a sensor insert is primarily intended for running or other sporting activities in order to gather useful data on how the user's feet are impacting the ground.

### BRIEF SUMMARY OF THE INVENTION

A problem with such sensor inserts is that they are thin flexible components which are subjected to many repeated shear loadings as a user runs. This creates a problem that the sensor will tend to be pushed along the shoe and will fold over on itself. This effectively renders it inoperable and means that the wearer has to stop their activity, remove the shoe and reposition the sensor. Further, the folding of the sensor insert can cause damage to the sensors.

This problem could be solved by increasing the thickness of the sensor insert. However, the insert is designed to be used in a shoe beneath the normal inner sole of the shoe. Increasing the thickness of the sore insert is therefore undesirable as there is limited space within the shoe. Further, integrating the sensor insert into the inner sole is also undesirable as the intention is that the sensor insert is supplied as standalone component which can be used with a variety of shoes and can be transferred from one shoe to another as necessary.

According to one aspect of the present invention there is provided a sensor insert for a shoe.

With this insert, the upper face with the low coefficient of friction minimizes the amount of shear force being transferred from the foot via the inner sole in the shoe to the sensor insert, while the lower face of the higher coefficient of friction resists any movement imparted to the insert from the foot.

Preferably, the use of a hock and loop (i.e. Velcro™) connector for attaching the flexible connection portion to the inside of the shoe provides an anchor point for the sensor insert thereby imparted further stability to the sensor insert. The insert is still very easy to remove from the shoe and transfer to another shoe.

The lower layer is preferably formed of a transparent material such that the sensors are visible through it. This allows a user to inspect the sensors to check for any damage.

The upper layer is preferable formed of a material with a dynamic coefficient of friction between 0.35 and 0.65, preferably 0.35 to 0.55 and more preferably 10 0.35 to 0.45. Preferably, the upper face is a PET fiber layer.

The lower face preferably has a dynamic coefficient of friction of between 0.75 and 1.2, preferably 0.85 and 1.2 and more preferably 0.95 and 1.2. Preferably the lower face is silicone. Silicone is a difficult material to bond to other materials so that the lower layer is preferably a co-formed laminate of silicone and PET. The silicone provides the lower face with the high coefficient of friction, while the PET provides a layer which can readily be bonded to the central layer. The central layer is preferably formed of two bonded PET sublayers between which the sensors are sandwiched. This can readily be bonded to the PET layers of the upper and lower layers.

The sensor insert can be integrated into an inner sole. However, the preference is for it to be a separate layer. The present invention therefore also extends to a combination of a shoe comprising an inner sole, with a sensor insert according to the first aspect of the invention between the sole of the shoe and the insole.

### BRIEF DESCRIPTION OF THE DRAWINGS

An example of a sensor insert in accordance with the present invention will now be described with reference to the accompanying drawings, in which:

FIG. 1 is a schematic plan view of the senor insert;

FIG. 2 is a schematic cross-section of a typical portion of the insert; and

FIG. 3 is a cross-section through a shoe showing how the insert is integrated in the shoe.

### DETAILED DESCRIPTION OF EXEMPLARY EMBODIMENTS OF THE INVENTION

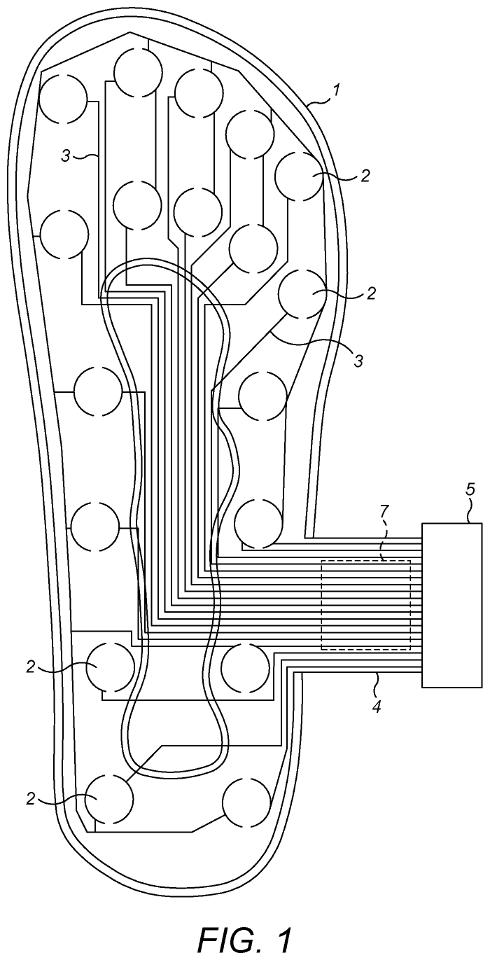

The sensor insert comprises a flexible planar portion 1 which has the shape of an inner sole of shoe as shown in FIG. 1. The insert comprises a plurality of sensors 2 which are force sensitive resistor (FSR) sensors which are described in our earlier GB2549513. These sensors may be of any suitable type and will not be described in greater detail here.

Each of the sensors is connected to a pair of electrical connections 3, each of which is routed through the insert to a connecting portion 4. This connecting portion 4 is an extension of the flexible planar portion 1, but has a flexible ribbon like structure via which all of the electrical connections 3 are connected to a terminal connection 5. This terminal connection 5 has a “puck” like structure and may be provided with a hook 6 via which it is attached to the rim of the shoe as shown in FIG. 3. This puck 5 may contain a number of components including memory, a battery, a wireless transmitter/receiver, a recharging mechanism which may either be wireless, or may be a connection port.

In broad terms, when a user runs, the sensors 2 sense the impact of each stride. The force of each impact is stored in the memory in the puck 5 and is later transmitted to an external device such as a smartphone or computer for further processing.

The present invention is concerned with the physical form of the insert which retains the insert reliably within the shoe.

One aspect of this concerns the use of a hook and loop type fastener such a Velcro® 7 which is provided on an outer surface of the flexible connecting portion 4, this may be designed to attach to the material of an inner face of the shoe. Alternatively the flexible connecting portion 4 is provided with one half of the hook and loop connector, while the other half of the connector is provided as an adhesive patch which is adhered to the inner face of the shoe by the user.

This ensures that the insert is firmly anchored at, at least one portion of the shoe.

A flexible planar portion 1 is also provided with a layered structure which assists in maintaining the insert in place as described below.

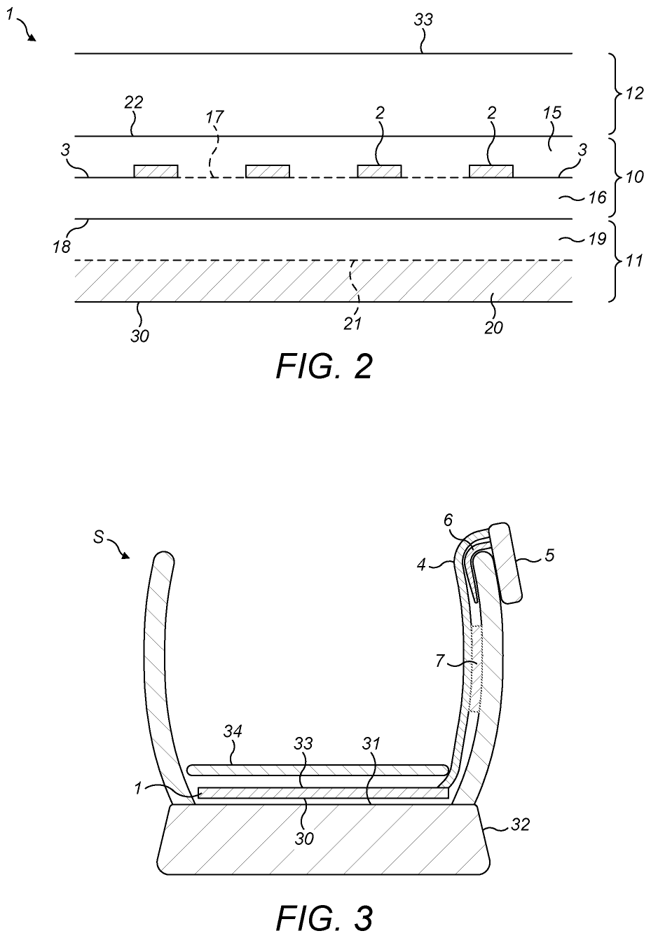

As shown in FIG. 2, the flexible planar portion 1 broadly comprises three layers, namely a central layer 10, a lower layer 11 and an upper layer 12.

The central layer 10 is formed of two PET sub-layers 15, 16 which are bonded to one another at a bonded interface 17 between which the sensors 2 and connectors 3 are sandwiched.

The central layer 10 has a bonded interface 18 with the lower layer 11. The lower layer 11 is formed of two sub-layers, namely an upper PET layer 19 and a lower silicone layer 20. These layer are co-formed, for example in a calendaring process to ensure that the interface 21 is adequately bonded, given that silicone is otherwise very difficult to attach to PET via an adhesive process. By contrast, the bonded interface 18 10 is between two PET layers which can form a strong adhesive bond.

The upper layer 12 is a PET layer with a bonded interface 22 to the central layer 10. This may be a microfiber layer and may be opaque as there is no need to be able to see through this layer. The lower layer 11 and the lower sub-layer 16 of the central layer 10 are transparent such that a user can see the pattern of sensors as shown in FIG. 1 as shown in FIG. 1 through the lower layer of the insert. Not only does this give the user an appreciation of the manner in which the insert is constructed, and therefore how they can expect it to function, it also allows them to visually inspect the sensors 2 and connectors 3 to ensure that they are undamaged.

The lower face 30 of the insert is formed of silicone and therefore has a high coefficient of friction. This provides a good grip between the lower face 30 and an upper face 31 of the sole 32 of the shoe. On the other hand, the upper face 33 of the insert 1 is made of PET which has a much lower co-efficient of friction. As a result of this, the inner sole 34 which is on top of the sensor insert 1 will reduce the transmission of shear loads to the flexible planar portion 1 as, instead, the insole 34 will tend to slide to a small extent over the upper surface 33. This, together with the attachment via the hook and loop fastener 7 provides a robust way of holding the insert in place. However, it is still very easy to transfer the insert to a different shoe.

What is claimed is:

1. A senor insert for a shoe, the insert comprising a flexible planar portion having a shape of an inner sole for a shoe, the flexible planar portion comprising:
a central layer containing a plurality of pressure sensors for sensing the pressure applied by the foot and electrical connections to the sensors;
a lower layer having a lower face arranged to face the sole of a shoe and having a first coefficient of friction; and
an upper layer having an upper face via which a foot is arranged to impact the insert and having a second coefficient of friction which is lower than the first coefficient of friction.

2. The sensor insert according to claim 1, further comprising a flexible connection portion extending from the flexible planar portion, the electrical connector extending along the flexible connecting portion to a terminal connection, the flexible portion having a hook and loop connector for attachment to the inside of a shoe.

3. The sensor insert according to claim 1, wherein the lower layer is formed of a transparent material such that the sensors are visible through it.

4. The sensor insert according to claim 1, wherein the upper layer is formed of a material with a dynamic coefficient of between 0.35-0.65, preferably 0.35-0.55, and more preferably 0.35-0.45.

5. The sensor insert according to claim 4, wherein the upper layer is a PET layer.

6. The sensor insert according to claim 1, wherein the lower layer is formed of a material with a dynamic coefficient of friction of between 0.75-1.2, preferably 0.85-1.2, and more preferably 0.95-1.2.

7. The sensor insert according to claim 6, wherein the lower layer is silicone.

8. The sensor insert according to claim 1, wherein the lower layer is a co-formed laminate of two different materials.

9. The sensor insert according to claim 8, wherein the lower layer is a co-formed laminate of silicone and PET.

10. The sensor insert according to claim 1, wherein the upper and lower layers are bonded to the central layer by an adhesive.

11. A shoe comprising an inner sole with a sensor insert according to claim 1 between the shoe and the insole.
